(19) United States
(12) Patent Application Publication
(10) Pub. No.: US 2023/0313462 A1
(43) Pub. Date: Oct. 5, 2023

(54) METHODS FOR ANALYZING PAPER AND IMPROVING THE EFFECTIVENESS OF PAPER ADDITIVES

(71) Applicant: SOLENIS TECHNOLOGIES, L.P., Wilmington, DE (US)

(72) Inventors: Matthew Nicholas, Avondale, PA (US); Terry Lynn Bliss, Marietta, GA (US); Tiffany Bohnsack, Avondale, PA (US)

(73) Assignee: SOLENIS TECHNOLOGIES, L.P., Wilmington, DE (US)

(21) Appl. No.: 18/193,694

(22) Filed: Mar. 31, 2023

Related U.S. Application Data
(60) Provisional application No. 63/362,304, filed on Mar. 31, 2022.

(51) Int. Cl. D21G 9/00; G01N 1/42; D21H 23/66; D21H 21/18

(52) U.S. Cl. CPC D21G 9/0045; G01N 1/42; D21H 23/66; D21H 21/18; G01N 2001/2873

(57) ABSTRACT

Methods of analyzing a sheet of paper, and methods of preparing paper are provided. In an exemplary embodiment, a method for preparing paper includes applying a paper additive to a first sheet of paper using an initial production technique. The first sheet of paper is sectioned into a first section and a second section, where the first sheet of paper has a top surface defined in an X-Y plane, where a Z axis is perpendicular to the X-Y plane, and where the first and second sections are defined at different positions along the Z axis. A paper additive concentration is measured in the first and second sections, and the initial production technique is adjusted to a subsequent production technique to influence a paper additive concentration profile along the Z axis.

### CROSS-REFERENCE TO RELATED APPLICATIONS

This application claims the benefit of U.S. Provisional Application No. 63/362,304, filed Mar. 31, 2022, the contents of which are incorporated herein in its entirety.

### TECHNICAL FIELD

The present disclosure relates to the field of paper analysis and applying additives to paper. More particularly, the present disclosure relates to methods for improving the effectiveness of paper additives in paper by measuring the distribution of the paper additive in the Z direction of the sheet of paper.

### BACKGROUND

In paper manufacturing, additives are introduced into the papermaking process to improve paper properties. For example, known additives improve paper strength, drainage properties, retention properties, and so on.

In a conventional papermaking machine, pulp is prepared for papermaking in a stock preparation system. Chemical additives, dyes, and fillers are sometimes added into the thick stock portion of the stock preparation system, which operates at a consistency of from 2.5 to 5% dry solids; additives may be added into the blend chest, the paper machine chest, a pulp suction associated with either of these chests, or other locations. In the thin stock circuit of the stock preparation system, the pulp is diluted from a consistency of 2.5 to 3.5% to a consistency of from 0.5 to 1.0% dry solids prior to passing through the thin stock cleaners, screens, an optional deaeration system, and approach flow piping. During or after this dilution, additional chemical additives may be added to the pulp, either in a pump suction, or in the headbox approach flow piping. Addition of chemical additives in the thick stock or the thin stock portions of the stock preparation system would be considered “wet-end addition” as used herein.

The fully prepared stock slurry, at from 0.5 to 1.0% dry solids consistency, is typically pumped to the headbox, which discharges the stock slurry onto a moving continuous forming fabric. The forming fabric may have the form of a woven mesh. Water drains through the forming fabric and the fibers are retained on the forming fabric to form an embryonic web while traveling from the headbox to the press section. As water drains away, the water content of the embryonic web may drop from 99 to 99.5% water to 70 to 80% water. Further water may be removed by pressing the wet web with roll presses in a press section, from which the wet web may exit with only from 50 to 60% water content (that is, a consistency of from 40 to 50% dry solids). Further water is typically removed from the web by evaporation in a dryer section, from which the web may exit with a consistency of from 90 to 94% dry solids. The sheet may then be calendered to improve the surface smoothness of the sheet, and to control the sheet thickness or density to a target value. The sheet is typically then collected on a reel.

As explained above, chemical additives, such as strength agents, may be introduced into the pulp within the stock preparation section, in what is known as “wet-end addition”. In some cases, strength agents may also be added via either spraying onto the wet web in the forming section, or by using a size press to apply the additives to the dry sheet. Spray application and size press addition of additives are optional.

In wet end applications, the chemical additives are distributed throughout the web and the retention of the chemical additives varies depending on the papermaking system and the chemistry being applied. There are additional considerations with wet end application of additives such as deposits on the forming fabric and other surfaces within the forming section, and potential cycle up issues (accumulation of wet end additives within the recirculated water due to poor fixation of the additives to the fibers). Spray application can be somewhat problematic due to accumulation of overspray on nearby surfaces and the plugging of the spray nozzles. Size press applications are not performed on the wet end of the papermaking machine and do not have the advantages of applying chemistry to a wet sheet prior to or during formation.

Further, chemical additives applied via traditional wet end application typically provide relatively uniform distribution of additives throughout a Z direction of the web (i.e., in a direction perpendicular to the surface of the web), which may be desirable, or may result in less additive in some Z direction locations within the sheet than desired. Thus, the wet end approach is not targeted to specific locations within the paper and can result in some cost inefficiencies in the chemistry application.

Some paperboard products are formed from multiple plies. The individual plies may advantageously be comprised of different types of fiber. This may be done to improve the properties of the sheet, or for cost savings reasons. In a three-ply sheet, the plies may be identified as the top ply (usually the preferred printing surface), the middle ply, and the back ply, which may or may not be printed. Typically the fibers used in the middle ply may be less costly or higher in bulk due to lack of bleaching or due to less refining or due to the fiber species or pulp production process, while the fibers in the top ply may be brighter and may produce a smoother printable surface. The back ply may be somewhat in between the cost and characteristics of the top and middle ply, or it may be very similar to the top ply if both sides are to be printed. Typically, the mass per unit area of the top ply and the back ply is minimized, to reduce the total cost. Typically, the middle ply has more mass per unit area than the top or back ply, especially if the sheet is exceptionally thick. Typically, all broke from the production process is sent to the middle ply, to preserve the appearance and printing qualities of the top ply, and, in some cases, the back ply.

There are many ways to produce sheets with separate stock characteristics in the various plies, including specialized headboxes which have separate inlets for the separate stocks, and vanes within the headbox that keep the stocks separate until they discharge from the headbox toward the forming fabric. This method is sometimes called “wet on wet” forming and has been well known by those skilled in the art for at least 35 years. Such a forming technique produces very good bonding between the plies, but the layer purity is not as good as preferred, and the drained waters from the different plies are generally mixed, which can cause some process problems during the reuse of the drained water in the forming section. This is especially true when there are large differences in the brightness of the top ply or the top and back ply, relative to the middle ply.

Another method well known to those skilled in the art is the use of a secondary headbox, which can apply a top ply onto a base or center ply while the base or center ply is at about 8 to 10% solids on the forming table. This method is sometimes called “wet on dry” multi-ply forming, since the base or middle ply has been partially dewatered prior to application of the very low consistency stock that will become the top ply. Such a forming technique typically provides better layer purity, and reasonably good bonding between the plies, but the water from the top ply is still somewhat mixed with the base or middle ply water as the combined sheet drains. The secondary headbox method of forming multi-ply (usually two ply) sheets has also been widely practiced for many years.

Yet another widely practiced method of forming multi-ply sheets is by producing a top ply on a papermaking former, and a middle ply on a second papermaking former. Occasionally, multiple middle plies may be produced on multiple separate papermaking formers. Yet another separate papermaking former may be used to produce a back ply. The plies are bonded together by lightly pressing one ply into another with a “combining roll” at about 8 to 12% solids after which the sheet may be further dewatered by application of additional vacuum to the combined sheet. Such papermaking forming sections are well known to those skilled in the art, and the technique may be called “dry on dry” forming, because the plies are separately dewatered to from 8 to 12% solids before they are combined. This method of forming produces exceptionally good layer purity, and also provides for the best separation of the water systems of the named plies. It is also known to those skilled in the art that the “dry on dry” forming technique has less effective bonding between the various plies, which sometimes results in delamination in the ply bond area during printing.

Ply bonding can be improved in multi-ply formed sheets, and particularly in “dry on dry” formed multi-ply sheets, by spraying a suspension of uncooked starch on one of the ply surfaces where ply bonding is insufficient. The uncooked starch is in the form of small particles which are retained by filtration on the application surface of the ply. The particles of uncooked starch absorb water over time, particularly as the sheet heats up in the dryer section, and with sufficient moisture and temperature, will gelatinize and form an adhesive bond between the fibers of the plies it contacts, thus improving ply bonding.

It is understood that if a unique stock composition is to be provided to different plies of a multi-ply sheet, a separate stock preparation system is required for each ply. The need for separate top ply, middle ply, and back ply stock preparation and forming sections make this multi-ply sheet forming method complex and capital intensive compared to sheets with only one ply, or with uniform composition in two or more of their plies.

Different types of paper additives may also be applied in addition to, or in place of, the application of uncooked starch on a surface of one or more of the separate plys. For example, a strength additive may be added for localized strength improvement. A drainage additive or a retention additive may also be applied with, or in place of, the uncooked starch and/or strength additive. These paper additives typically migrate within the paper during the dewatering processes, pressing processes, etc. Improvements of the efficiency of the paper additives is desired to improve the performance of the paper, and/or to reduce the amount of the paper additive being used to produce a desired result. Furthermore, other desirable features and characteristics of the present disclosure will become apparent from the subsequent detailed description and the appended claims, taken in conjunction with the accompanying drawings and this background.

### BRIEF SUMMARY

Methods of analyzing a sheet of paper, and methods of preparing paper are provided. In an exemplary embodiment, a method for preparing paper includes applying a paper additive to a first sheet of paper using an initial production technique. The first sheet of paper is sectioned into a first section and a second section, where the first sheet of paper has a top surface defined in an X-Y plane, where a Z axis is perpendicular to the X-Y plane, and where the first and second sections are defined at different positions along the Z axis. A paper additive concentration is measured in the first and second sections, and the initial production technique is adjusted to a subsequent production technique to influence a paper additive concentration profile along the Z axis.

A method of analyzing a sheet of paper is provided in another embodiment. A first sheet of paper is attached to a microtome, where the first sheet of paper has a top surface defined in an X-Y plane. The microtome is configured to slice the first sheet of paper along a Z axis that is perpendicular to the X-Y plane. The first sheet of paper is sliced into a first section and a second section that are defined at different positions along the Z axis. The paper additive concentration is measured in the first and second sections.

A method of preparing paper is provided in yet another embodiment. A paper additive is applied to the first sheet of paper and a second sheet of paper using an initial production technique. The first sheet of paper is attached to a microtome, where the first sheet of paper has a top surface defined in an X-Y plane. The microtome is configured to slice the first sheet of paper along a Z axis that is perpendicular to the X-Y plane. The first sheet of paper is sliced into a first section and a second section with the microtome, where the first and second sections are defined at different positions along the Z axis. A reference sheet of paper is produced using a reference production technique, where the reference production technique is the same as the initial production technique with the exception that the reference production technique is free of a paper additive addition step, so the reference sheet of paper is free of the paper additive. The nitrogen concentration of the reference sheet of paper is measured, and the nitrogen concentration of the first section is measured. A paper additive induced nitrogen concentration in the first section is determined by reducing the nitrogen concentration measured in the first section to account for the nitrogen concentration measured in the reference sheet of paper. A paper additive concentration in the first section is determined based on the paper additive induced nitrogen concentration. A selected paper property is measured on the second sheet of paper, where the paper additive influences the selected paper property. The above steps are repeated, with the exception of applying the paper additive to a subsequent sheet of paper using a subsequent production technique that is different than the initial production technique, and where the paper additive concentration is measured in the subsequent sheet of paper. An improved paper production technique is then obtained as shown by comparisons of the first sheet of paper to the subsequent sheet of paper.

### BRIEF DESCRIPTION OF THE DRAWINGS

A more complete understanding of the subject matter may be derived from the following detailed description taken in conjunction with the accompanying drawings, wherein like reference numerals denote like elements, and wherein:

FIG. 1 is a schematic of a multi-ply papermaking apparatus in accordance with various embodiments;

FIG. 2 is a block diagram of an exemplary embodiment for improving the performance of a paper additive;

FIG. 3 is a perspective view of a microtome slicing a sheet of paper;

FIG. 4 is a graph illustrating filler distribution in handsheets;

FIG. 5 is a pair of graphs illustrating filler distribution in uncoated sheets by different forming unit types;

FIG. 6 is an exemplary embodiment of a confocal laser scanning microscope;

FIG. 7 is a graph illustrating an exemplary embodiment of a nitrogen concentration vs. sheet depth in a multi-ply sheet of paper; and

FIG. 8 is a graph illustrating the variation in nitrogen concentration vs. sheet depth to demonstrate the accuracy of the techniques described herein;

FIG. 9 is a graph illustrating nitrogen concentration vs. sheet depth for two different application levels for a paper additive; and

FIG. 10 is a graph illustrating the nitrogen concentration vs. sheet depth for several samples to further demonstrate the accuracy of the techniques described herein.

### DETAILED DESCRIPTION

The following detailed description is merely illustrative in nature and is not intended to limit the embodiments of the subject matter or the application and uses of such embodiments. As used herein, the word “exemplary” means “serving as an example, instance, or illustration.” Thus, any embodiment described herein as “exemplary” is not necessarily to be construed as preferred or advantageous over other embodiments. All of the embodiments described herein are exemplary embodiments provided to enable persons skilled in the art to make or use the systems and methods defined by the claims. Furthermore, there is no intention to be bound by any expressed or implied theory presented in the preceding Technical Field, Background, Brief Summary, or the following Detailed Description. For the sake of brevity, conventional techniques and compositions may not be described in detail herein.

As used herein, “a,” “an,” or “the” means one or more unless otherwise specified. The term “or” can be conjunctive or disjunctive. Open terms such as “include,” “including,” “contain,” “containing” and the like mean “comprising.” The term “about” as used in connection with a numerical value throughout the specification and the claims denotes an interval of accuracy, familiar and acceptable to a person skilled in the art. In general, such interval of accuracy is ±ten percent. Thus, “about ten” means nine to eleven. All numbers in this description indicating amounts, ratios of materials, physical properties of materials, and/or use are to be understood as modified by the word “about,” except as otherwise explicitly indicated. As used herein, the “%” described in the present disclosure refers to the weight percentage unless otherwise indicated.

A sheet of paper is generally described herein using a cartesian coordinate system where the length and width of the sheet of paper are defined in the X-Y plane, and the thickness of the sheet of paper is defined in the Z plane. As such, the sheet of paper has a top surface that is defined in the X-Y plane. In general, the sheet of paper has a greater length and width than the depth in the Z direction. Of course, it is possible to cut a sheet of paper such that the X or Y direction are smaller than the width in the Z direction. In this description, a sheet of paper has a significantly varying composition or concentration of certain components in the Z direction, and the overall composition and concentration of components is relatively consistent from one location to the next in the X and Y directions.

Embodiments of the present disclosure relate to testing of a sheet of paper to determine variations of a paper additive concentration in the Z direction. The paper additive may be a wide variety of different possible components, including dry strength additives, wet strength additives, retention aids, sizing agents, binders, coating agents, optical brighteners, biocides, dyes, etc. A production technique is utilized for the paper, where the paper additive is applied to a surface of the paper during production. The paper additive may be applied by spraying, foam application, rolling, or other techniques, but the paper additive is applied to a surface such that the concentration of the paper additive may not be consistent throughout the thickness of the paper. In a multi-ply sheet of paper, the paper additive may be applied to an internal or external surface of one or more of the plies.

In an exemplary embodiment, a method for manufacturing a multi-ply paper sheet is provided. However, the techniques and methods described herein may be utilized for both single-ply and multi-ply paper. The description of the multi-ply paper sheet also provides a guide for single-ply paper sheets, as understood by one skilled in the art.

Application of paper additives to a surface of the wet web via foam application, spray application, roll-on, or other techniques can be advantageous in that the paper additive is applied to the wet end, as with traditional approaches, but some of the typical disadvantages are avoided. Surface application can be expected to have better paper additive retention, thereby reducing or avoiding deposits, and application to the wet web surface allows some penetration into the web. Embodiments using surface application of paper additives have some advantages over the standard practices in terms of efficiency, cost, and targeted application, where the paper additive may be placed at the point within the depth of the paper where it is the most beneficial.

The paper additive may be pulled into the web via vacuum, or negative pressure force, which can provide multiple advantages over traditional approaches. For example, the application to the surface can be optimized to provide better retention in the web as compared to conventional wet end applications. Additionally, the application to the web surface allows for tunable penetration into the web and a controlled distribution from one surface as opposed to an even distribution throughout the Z direction of the web.

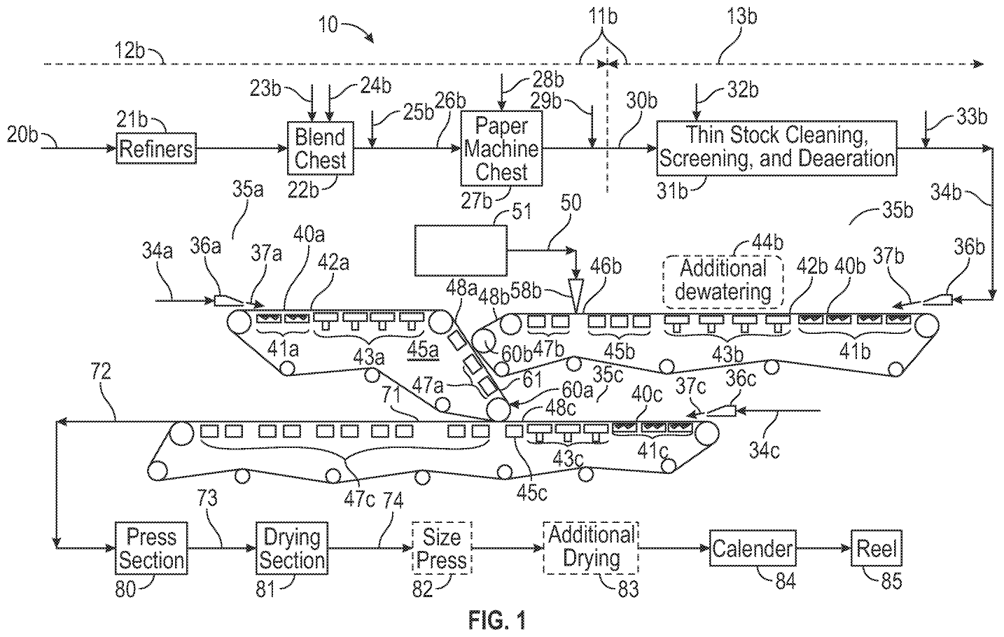

A schematic of a device 10 for the formation of a three-ply sheet of paper using a “dry on dry” method, and for applying a paper additive to a wet embryonic web surface is shown in FIG. 1. The device 10 includes a middle ply stock preparation section 11b which includes a middle ply thick stock circuit 12b and a middle ply thin stock circuit 13b. In this figure, the flow of a middle ply thick stock component 20b is illustrated using solid arrows. In an embodiment, the middle ply thick stock section 12b comprises one or more middle ply refiners 21b configured to improve fiber-fiber bonding in the middle ply thick stock component 20b by making fibers of the middle ply thick stock component 20b more flexible and by increasing their surface area through mechanical action applied to the middle ply thick stock component 20b at a consistency of from about 2.0 to about 5.0% dry solids. In an embodiment, subsequent to the refiners, the middle ply thick stock component 20b enters a middle ply blend chest 22b. In the middle ply blend chest 22b, the middle ply thick stock component 20b may optionally be blended with middle ply stock component or components 23b from other sources, for example, broke. Additionally, the middle ply thick stock component 20b may be blended with chemical additives 24b in the middle ply blend chest 22b. After exiting from the middle ply blend chest 22b, the middle ply thick stock components 20b and 23b may be diluted through the addition of water 25b in order to control the consistency of the middle ply thick stock components 20b and 23b to be within a pre-determined target range; the blended and consistency adjusted middle ply stock can now be called 26b. The middle ply stock 26b then enters a middle ply paper machine chest 27b where additional chemical additives 28b may be added. In an embodiment, as the stock exits from the middle ply paper machine chest 27b, the middle ply stock 26b is diluted with a large amount of water 29b to control the consistency of the middle ply stock 26b to be from about 0.5 to 1.0% dry solids as the middle ply stock 26b exits the middle ply thick stock circuit 12b. The middle ply stock 26b, with a consistency of from 0.5 to 1.0% dry solids, can now be called 30b as it enters the middle ply thin stock circuit 13b.

In an exemplary embodiment, within the middle ply thin stock circuit 13b, the middle ply stock 30b may pass through low consistency cleaning, screening, and deaeration devices. In exemplary embodiments, additional chemical additives 32b may be added to the middle ply stock 30b in any number of locations within the middle ply cleaning, screening, and deaeration area 31b, for example at location 33b in the approach flow piping 34b to the middle ply forming section 35b. The middle ply stock 30b can now be called 37b as it enters the mid ply forming section 35b. In exemplary embodiments, in the middle ply forming section 35b, a middle ply headbox 36b distributes the middle ply stock 37b onto a moving woven fabric (the middle ply “forming fabric”) 40b. In exemplary embodiments, the middle ply forming fabric 40b transports the middle ply stock 37b over one or more boxes of hydrafoils 41b, which serve to drain water from the middle ply stock 37b and thereby increase the consistency of the middle ply stock 37b to form an embryonic middle ply web 42b. In exemplary embodiments, when the embryonic middle ply web 42b has a consistency of from 2 to 3% dry solids, the embryonic middle ply web 42b then passes over one or more low vacuum boxes 43b, which are configured to apply a “low” vacuum to the embryonic middle ply web 42b in order to remove additional water from the web. The embryonic middle ply web 42b may also be dewatered further by an optional additional dewatering unit 44b mounted above the middle ply forming fabric 40b. The embryonic middle ply web 42b be may subsequently pass over one or more “high” vacuum boxes 45b, where a higher vacuum, i.e., stronger negative pressure, force removes additional water until the embryonic middle ply web 42b has a consistency of from 6 to 12% dry solids. The wet middle ply web, no longer embryonic, is now referred to as 46b.

In an exemplary embodiment, a paper additive 50 is applied to a surface of the wet middle ply web 46b. In an exemplary embodiment, the paper additive 50 may be combined with other materials for application to the middle ply web 46b. For example, water, foaming agents, surfactants, air for foam applications, etc. may be added with the paper additive 50 at a paper additive application site 58b. The paper additive application site 58b may be located between the high vacuum box 45b and a post-application high vacuum box 47b. The vacuum created by the post-application high vacuum box 47b following the paper additive application site 58b draws the paper additive 50 into the wet middle ply web 46b. The vacuum treated middle ply web (now called 48b) with the added, surface applied paper additive 50, is also typically at a somewhat higher consistency, from 8 to 12%, due to the influence of vacuum from the post-application high vacuum boxes 47b.

The above description is of the middle ply production process of device 10 (middle ply stock preparation system 11b, middle ply forming system 35b, and the paper additive application site 58b). It acts in conjunction with a top ply former 35a and back ply former 35c (comparable to middle ply former 35b). The top ply former 35c and the back ply former 35c are supported by corresponding top and back ply stock preparation systems (not shown in FIG. 1.). The wet top ply web 48a produced by the top ply former 35a is merged with the vacuum treated middle ply web 48b by combining roll 60b, which transfers the wet middle ply web to the top ply wet web on the top ply forming fabric 40a between an initial top ply high vacuum box 45a and the final top ply high vacuum boxes 47a.

The wet top ply web 48a and the vacuum treated middle ply web 48b, called a combined top and middle ply web 61 when combined, is transferred to the wet back ply web 48c by combining roll 60a, which presses the combined wet top and middle ply web 61 to the wet back ply web 48c immediately following the back ply high vacuum box 45c and before the back ply subsequent high vacuum boxes 47c on the back ply former 35c. The web 71 is comprised of the combined wet top ply web 48a, the vacuum treated middle ply web 48b, and the wet back ply web 48c. The combined wet web 71 may be further dewatered by the back ply subsequent high vacuum boxes 47c on the back ply former 35c to about 20 to 25% solids, and is now called the web 72.

The combined web 72 enters the pressing section 80, where press rolls press additional water from the wet web 72. The wet web 72 exits the pressing section 80 with a consistency of about 40 to 55% dry solids and is then called web 73. Wet web 73 enters a drying section 81, where heated dryer cylinders heat the web 73 and evaporate additional water from the web 73. The wet web 73 is dried to 90 to 94% solids within the drying section and is now called the dry sheet 74. After the drying section 81 the dry sheet 74 may go directly to the calendar 84 and reel 85, or it may be treated with a surface size in the optional size press 82; if so treated, it is then dried again with additional dryers 83. Following the drying section 81 or optionally size press 82 and additional drying 83, the dry sheet 74 may be treated with a calender 84 to improve surface smoothness and control sheet thickness, then the sheet may be reeled by a reel device 85.

It should be understood that the description of the middle ply stock preparation section 11b and middle ply forming section 35b which produces the vacuum treated middle ply web 48b, is also a good general description of the top ply and back ply stock preparation systems (not shown in FIG. 1). Further, the description of the middle ply forming section 35b is also a good general description of the top ply former 35a and the back ply former 35c, respectively. Each numbered item in each web forming system are correspondingly numbered, with the suffix “b” applied to the components of the middle ply forming section 35b, the suffix “a” applied to the correspondingly numbered components of the top ply system, and the suffix “c” applied to the correspondingly numbered components of the back ply former 35c. For example, top ply headbox 36a corresponds to middle ply headbox 36b and back ply headbox 36c, and so on.

It is also clearly understood by those skilled in the art that a number of variations in the details may differ from one manufacturing plant location to another, yet the same purpose is accomplished and hence such variations are contemplated as part of the system described and claimed herein. For example, middle ply thick stock circuit 12b shows refiners acting on middle ply thick stock component 20b, but not on an additional stock component or the middle ply stock component 23b. In some cases, other stock components may be blended with the middle ply thick stock component 20b before the middle ply refiners 21b and co-refined with the middle ply thick stock component 20b. There may be fewer or more boxes of hydrofoils 41b, low vacuum boxes 43b, or high vacuum boxes 45b, and the location for the addition of the paper additives 50 may be changed. Additional dewatering units 44b, for example, are identified as optional. The paper additive system 51 may position the paper additive application site 58b at any accessible location, and the choice for the location of the paper additive application site 58b depends on the paper additive being used, and the desired results. For example, the paper additive application site 58b may be positioned in the front or back ply production process, or at more than one location. In some embodiments, there may be only two plies and in other embodiments there may be three or more plies. It is also possible to have a single ply in some embodiments. The paper additive 50 may be advantageously applied between any two adjacent plies to improve a property of the paper, such as enhance ply bonding and other Z direction strength properties. The size press 82 combined with additional drying 83 are likewise shown as optional—they may be present in some cases and absent in other cases, within the scope of the system described herein. Many other similar variations may be within the scope of the system described herein.

Further, adjustment of the process variables (amount of paper additive 50 per unit of sheet area, time and strength of vacuum application before and after the addition of paper additive 50, concentration and constitution of the solution that includes the paper additive that is prepared in the paper additive system 51, location of the paper additive system 51, ply thickness, ply % dry solids at the time of the paper additive addition, and many other variables) can allow the distribution of the paper additive to be altered. This allows for control of the paper additive distribution within the Z direction of the paper, or the paper additive concentration profile.

It is understood that the system described herein is not limited to the exact configuration as shown in FIG. 1. A wide variety of papermaking systems are possible, and the example in FIG. 1 provides a reference for general understanding.

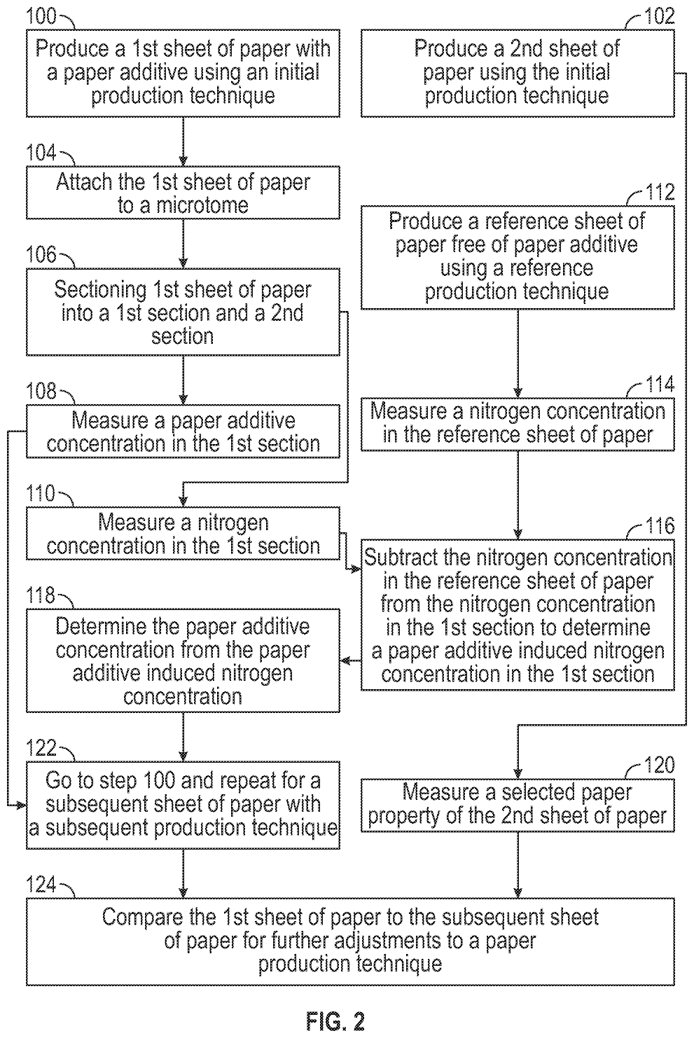

Reference is made to the flow chart in FIG. 2, with continuing reference to FIG. 1. FIG. 2 represents an exemplary embodiment, but variations are possible within the scope of this description. Some variations are described herein, so the embodiment described in FIG. 2 is not intended to limit this description. An initial production technique is used to produce a first sheet of paper in step 100, and the same initial production technique is used to produce a second sheet of paper in step 102. The initial production technique includes the addition of a paper additive 50, so the first sheet and the second sheet have the same amount of paper additive added in the same manner. The paper additive 50 may be added to a surface of the first and second sheets of paper, where the surface may be a top surface, a bottom surface, or an interior surface that is between plies within the depth of the sheets of paper. In embodiments where the paper additive 50 is applied to a surface, the concentration of the paper additive may vary throughout the Z direction, i.e. the depth, of the paper. Alternatively, the paper additive 50 may be added to one or more of the plies in a multi-ply sheet of paper, so that the paper additive may migrate from the ply to which it was added to another ply. In any event, the initial production technique produces a sheet of paper that has a non-uniform distribution of paper additive 50 through the Z direction, or through the depth, of the paper. A paper additive concentration profile provides a representation of the concentration of the paper additive along the depth of the paper. The first sheet is intended for destructive testing to determine the paper additive concentration profile through the Z direction, and the second sheet is intended for measuring a selected paper property, where the selected paper property in influenced by the paper additive, as will be described more fully below. For example, in embodiments where the paper additive is a strength additive, the second sheet may be tested for strength.

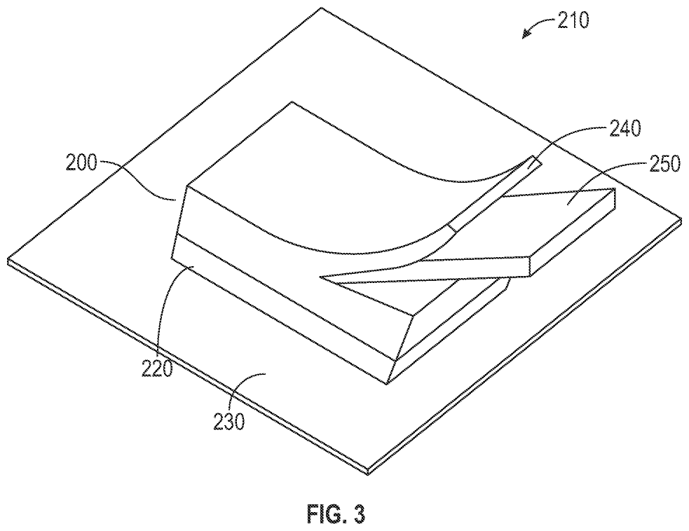

In an exemplary embodiment, the first sheet of paper 200 is attached to a microtome 210 in step 104, as illustrated in FIG. 3 with continuing reference to FIGS. 1 and 2. In an exemplary embodiment, the first sheet of paper 200 is attached to the microtome 210 with an attachment fluid 220, where the attachment fluid 220 is frozen such that the first sheet of paper 200 is frozen in place to the microtome 210. As such, the microtome 210 may be a cryostat microtome that utilizes freezing temperatures to freeze the attachment fluid 220. The attachment fluid 220 may be a tissue freezing medium. It has been discovered that tissue freezing medium does not penetrate into the first sheet of paper 200, or only penetrates to a small degree, so the internal constitution of the first sheet of paper 200 is not influenced by the tissue freezing medium, or is only influenced to a small degree. Furthermore, the overall depth of the first sheet of paper is not altered by the adsorption of the tissue freezing medium, or is only altered to a small degree. Changes to the depth of the first sheet of paper 200 may complicate the development of a paper additive concentration profile through the depth of the first sheet of paper, so minimal or non-existent adsorption of the tissue freezing medium facilitates further analysis. In alternate embodiments, the first sheet of paper may be attached to the microtome using alternate techniques. For example, adhesive may be used, a plate applying pressure from the top may be used, vacuum may be used between a mounting plate 230 of the microtome 210 and the first sheet of paper 200, or other techniques. If adhesive is used, the adhesive should be selected to avoid contamination of the first sheet of paper 200 with anything that may influence the measurement of the paper additive, as discussed further below.

The first sheet of paper 200 is sectioned into a first section 240 and a second section (not illustrated) in step 106. In an exemplary embodiment, the first sheet of paper is sliced by the microtome 210. However, in alternate embodiments, the first sheet 200 can be sectioned using an alternate technique, provided that a first section 240 and a second section are produced. For example, the first sheet of paper 200 may be sequentially ground to different depths to produce the first and second sections 240, differential freezing techniques may be utilized, and other possible techniques exist for sectioning the first sheet of paper 200 at different positions along the Z axis. The microtome 210 may include a razor 250 for slicing the first sheet of paper 200, but a knife or other cutting device(s) may alternatively be utilized. In an exemplary embodiment, the first sheet of paper 200 is sectioned into more than two sections. For example, the first sheet of paper 200 may be sectioned into 2 sections, or 5 sections, 10 or more sections, or almost any desirable number that is within the capabilities of the microtome 210 or the sections method utilized. The greater the number of sections, the more detailed and accurate the paper additive concentration profile may be. The sectioning of the first sheet of paper 200 into different sections results in a destructive test, because the first sheet of paper is no longer present as a whole, and cannot be tested for properties as a whole, but only as portions of the whole. The different sections of the first sheet of paper 200 may be present as a solid slice, or a ground section, or in other forms, as long as the contents are available for measurement.

A paper additive concentration is measured in the first section 240 in step 108. The paper additive concentration may be measured in a wide variety of manners. Many paper additives 50 are used in small quantities in paper, such as concentrations of less than 1%. Therefore, the measurement technique may need to be quite sensitive and reproducible. For example, some strength additives are used at concentrations of less than 1% by weight, based on a weight of the paper, and may be used at concentrations as low as, or lower than, 0.1% by weight. In an exemplary embodiment, the paper additive may be extracted from the first sections 240 with a known quantity of an extracting fluid, and then the concentration of the paper additive may be measured in the extracting fluid. For example, gas chromatography (GC) may be used, which may be combined with mass spectrometry (MS). Alternatively, liquid chromatography (LC) may be used. For quantitative testing, the mass of the first section 240 and the quantity of extracting fluid used and recovered may be recorded. The paper additive would need to be soluble in the extracting fluid. Other measurement techniques are also possible.

In order to avoid issues with incomplete extraction, alternative measurement techniques may be utilized in addition to, or in place, of those mentioned above. For example, the paper additive 50 may include an element that can be tested for, where the concentration of the element can be used to determine the concentration of the paper additive. Steps 110, 112, 114, 116, and 118 provide one embodiment of a technique for measuring the paper additive concentration, as in Step 108. For example, many strength additives include the element nitrogen. Exemplary strength additives include, but are not limited to, various types polyacrylamide, polyamines, and polyamidoamines. Other additives may have other elements present, such as metals in certain pigments. Once the percentage of the molecular weight of the paper additive 50 that results from nitrogen is known, the concentration of the paper additive 50 can be determined from the concentration of the nitrogen due to the paper additive. However, paper includes many different types of compounds, and some of those compounds include nitrogen. Therefore, the amount of nitrogen that is normally present in the paper should be accounted for. As such, in an exemplary embodiment, a concentration of nitrogen in the first section is measured, as noted in step 110 of FIG. 2.

A reference sheet of paper is produced using a reference production technique, as noted in step 112 of FIG. 2. The reference sheet of paper is used to account for the nitrogen that is present in the paper, but that is not from the paper additive 50. The reference production technique is the same as the initial production technique used for the first and second sheets of paper, with the exception that no paper additive 50 is added to the reference sheet of paper in the reference production technique. As such, the reference production technique is free of the paper additive addition step, and the reference sheet of paper is free of the paper additive 50. The same amount and type of fibers are used, the same amount and type of fillers are use, and the same amount of any additives that are not the paper additive 50 of interest are used. The reference sheet of paper is produced such that the only difference between the reference sheet of paper and the first sheet of paper is that the first sheet of paper includes the paper additive 50 of interest, and the reference sheet of paper does not.

The reference sheet of paper may be sectioned into comparable sections as the first sheet of paper 200 in some embodiments. For example, if a multi-ply paper is being investigated, the baseline amount of nitrogen may be different in the different plys. As such, the sections of the reference sheet of paper may be about the same number and thickness as the sections of the first sheet of paper 200. If the first sheet of paper 200 is sectioned into sections with varying thickness, the reference sheet of paper may be sectioned in the same manner. Comparable sections of the reference sheet of paper and the first sheet of paper 200 may be compared, as understood by one skilled in the art. For the sake of simplicity, this description will proceed with reference to a single-ply reference sheet, but it is to be understood that comparable sections may be used in alternate embodiments.

The nitrogen concentration in the reference sheet of paper is measured, as indicated in step 114 of FIG. 2. As mentioned above, this may also mean the nitrogen concentration of the different sections of the reference sheet of paper are measured. However, in alternate embodiments, the nitrogen concentration of the reference sheet of paper as a whole may be measured and used to determine the paper additive induced concentration of nitrogen in the first sheet of paper 200. In an exemplary embodiment, the nitrogen concentration of the reference sheet of paper (or the appropriate section of the reference sheet of paper) is then subtracted from the nitrogen concentration of the first section 240 to determine a paper additive induced nitrogen concentration in the first section 240, as noted in step 116 of FIG. 2. However, in an alternate embodiment, the mass of nitrogen for all the first sheet of paper sections are combined, and the mass of nitrogen for all the reference sheet of paper sections are combined (or the nitrogen in entire reference sheet of paper is measured, instead of in sections), and the percentage difference is used to estimate the paper additive induced nitrogen concentration in first section 240 and for every other section of the first sheet of paper 200 that is analyzed. For example, if the reference sheet of paper as a whole has 5% less nitrogen than the first sheet of paper 200 (i.e. nitrogen in first sheet of paper times 0.95=nitrogen in reference sheet of paper 200), the concentration of nitrogen in every section of the first sheet of paper 200 (including the first section 240) is reduced by 95% to determine the paper additive induced nitrogen concentration.

In yet another embodiment, the reference sheet of paper is sectioned, and the nitrogen concentration is measured for each section. This can then be plotted, such that a reference sheet of paper nitrogen concentration profile in the Z direction is produced. This may optionally be repeated, such that the reference sheet of paper nitrogen concentration provile in the Z direction is produced as the average of more than one reference sheet of paper. The measured nitrogen concentration for the first sheet of paper 200 may also be plotted, and more than one first sheet of paper 200 may optionally be used to produce average values for a measured first sheet of paper nitrogen concentration profile in the Z direction. The paper additive induced nitrogen concentration than can be determined by subtracting the reference sheet of paper nitrogen concentration profile in the Z direction from the measured first sheet of paper nitrogen concentration profile in the Z direction at each point along the graph, from a back to a top of the reference and first sheets of paper. Alternative techniques for determining the paper additive induced nitrogen concentration in the first section 240 may also be utilized. This step 116 may be repeated for each section of the first sheet of paper 200, or for each section of the first sheet or paper 200 that is analyzed if less than all the sections are analyzed. As such, the second section may also be analyzed for the nitrogen concentration, and the nitrogen concentration in the reference sheet of paper used to determine the paper additive induced nitrogen concentration in the second section.

The paper additive concentration from the paper additive induced nitrogen concentration is then determined, as noted in step 118 of FIG. 2. This may be done by dividing the paper additive induced nitrogen concentration by the percentage of the molecular weight of the paper additive that results from nitrogen. An element other than nitrogen may be tested for if the paper additive includes a different element in alternate embodiments. Nitrogen is used for some of the paper strength additives, because nitrogen is not expected to be as prevalent in the reference sheet of paper as other elements that could be used, such as carbon, oxygen, and hydrogen. The reduced prevalence of nitrogen may help improve the accuracy of the testing protocol.

The measurement of the paper additive concentration in the first section 240 can be repeated for the second section, and any other sections of the first sheet of paper 200, as mentioned above. This can produce a paper additive concentration profile along the Z axis of the first sheet of paper 200. The second sheet of paper is essentially the same as the first sheet of paper 200, so a selected property of the second sheet of paper is measured in step 120 of FIG. 2. The selected paper property is related to the paper additive 50, where the paper additive is utilized for the selected paper property. For example, if the paper additive 50 is a strength additive, the strength of the second sheet of paper can be measured. The measured selected paper property can then be correlated to the paper additive concentration profile to provide a greater understanding of how best to utilize the paper additive 50.

The nitrogen concentration in the first sheet of paper 200, and in the reference sheet of paper, can be measured using a variety of techniques. For example, pyrochemiluminescence can be used. In an alternate method, atomic adsorption can be used. Other measurement techniques may be utilized in alternate embodiments, or for different elements. The Example provided below describes a pyrochemiluminescence embodiment in greater detail. One skilled in the art can utilize the description in the Example provided below, or modify the technique as desired.

The initial production technique may then be modified to produce a subsequent production technique, where the subsequent production technique incorporates a change that will influence the paper additive concentration profile. For example, if the paper additive is water soluble, dewatering processes used to remove water from one or more of the plys, or from the paper as a whole in a single-ply sheet of paper, can be modified such that the paper additive moves more or less in the subsequent production technique as compared to the initial production technique. The process summarized in FIG. 2 can then be repeated by returning to Step 100, with the exception that the initial product technique in steps 100 and 102 is replaced with the subsequent production technique to produce a subsequent sheet of paper, as noted in step 122 of FIG. 2. This can produce a subsequent paper additive concentration profile, combined with a subsequent measurement of the selected paper property, so that the change in the paper additive concentration profile from the first sheet of paper 200 to the subsequent sheet of paper can be correlated with the change in the measured selected paper property. The entire process can then be repeated again if desired, so that the paper additive concentration profile can be optimized to produce the greatest effect on the selected paper property. The first sheet of paper is compared to one or more subsequent sheets of paper for further adjustments to a paper production technique, as indicated in step 124 of FIG. 2. This can inform the decision on how to produce the paper to improve the performance of the paper, or reduce the amount of paper additive needed to produce a desired effect. Once the paper production technique is optimized, an improved paper production technique can be developed and incorporated into a commercial paper production operation to commercialize the improvements elucidated by the methods and techniques discussed above.

### EXAMPLES

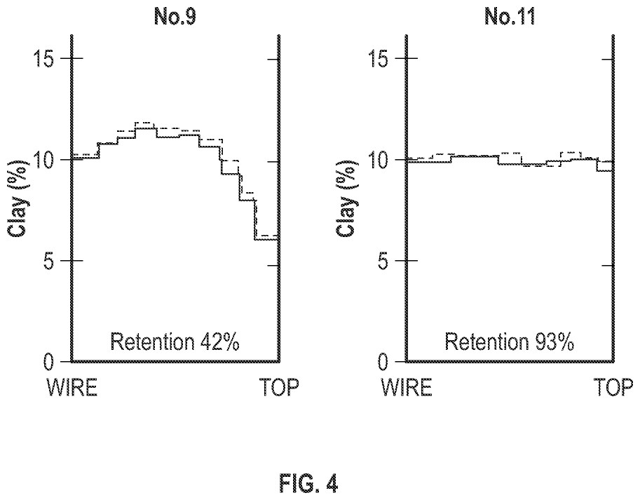

Fibrous substrates, such as paper, are generally described as a 2-dimensional material, albeit with distinctly different properties in the machine direction (MD) than in the cross-machine direction (CD), and sometimes with distinctly different properties on the wire than on the felt side. The former is due to preferential fiber alignment and stretch in the MD during manufacturing, while the latter is due to the combined effects of filtration within the sheet in the z-direction and washout of fines and filler on the wire side. Handsheets typically form with little or no turbulence over the forming fabric, such that filtration is dominant; the lower half of the sheet (closest to the wire) is higher in filler and fines than the upper half (the felt side). By comparison, table activity in commercially formed paper has a much greater influence on the portion of the sheet closest to the forming fabric, such that washout of fine material tends to dominate, resulting in a very different z-direction ash and fines distribution. The degree of this impact is exaggerated when the overall retention is low, as shown in FIG. 4

In FIG. 4, filler distribution in handsheets is graphically shown. The wire side filler content is higher than the felt side; this trend is exaggerated at low overall retention as shown on the left. A higher retention sheet is shown on the right.

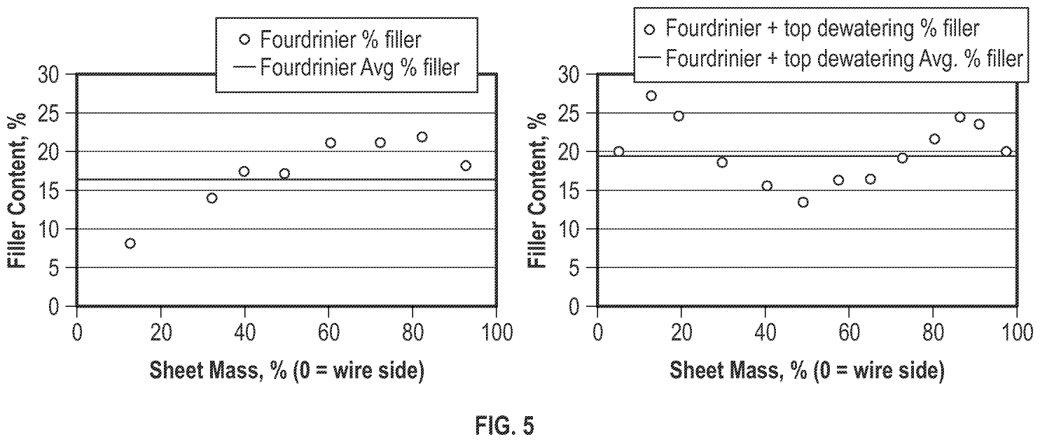

The forming process (one-sided or two-sided dewatering) also influences the filler and fines distribution, with two-sided dewatering producing a sheet with two “wire” sides and a characteristic “M” shaped filler distribution, as shown in FIG. 5. Other factors such as basis weight, fiber morphology, forming consistency, evenness of the water split and turbulence levels in two-sided dewatering may also influence these trends. In the case of multi-ply forming, each ply can be thought of as a separate sheet if the sheets are produced on mini-fourdriniers (“dry-on-dry forming”) since the filler and fines cannot readily move between plies because of a lack of turbulence after the combining point. Multi-layer headboxes (“wet-on-wet forming”) and secondary headboxes (“wet-on-dry forming”) may be somewhere between single-sheet forming and dry-on-dry forming. The general trends for fines and filler distributions within sheets formed on a variety of single-ply former types are well-documented.

FIG. 5 illustrated filler distribution in uncoated wood-free sheets that varies by forming unit type. The left graph results from a 75 grams per square meter (gsm) sheet made with a fourdrinier former, and shows a single peak curve, with more filler near the top (felt side) and substantial turbulence-based depletion close to the wire. The right graph results from an 80 gsm sheet made with a fourdrinier with top dewatering unit, and shows a two-peak curve, with reduced filler in the center due to migration outward and turbulence-based depletion at the wire surfaces. The two-peak form is characteristic of all sheets with two-sided dewatering.

Fines are chemically identical to fibers, but fines have a much higher specific surface area. The mostly cationic wet-end additives tend to associate more with fines than fibers based on their surface area, especially if added in the thin stock circuit, where the fines content is much higher because of the white-water recirculation. This suggests that charged wet-end additives may display a non-uniform distribution in the z-direction because they preferentially associate with the fines; however, the impact of this association may be small, especially when the overall fines retention is high.

Not all additives are added in the wet end. Starch and other chemical additives can be added in a size press or as a coating. Many mills add starch or synthetic dry strength additives onto a forming section via a shower or as a cascade. Furthermore, interest in adding strength and other additives using foam application is growing. When additives are applied as a foam on a sheet at 8-10% consistency, little time exists for static attraction and little turbulence is available to influence fines washout. Additives may vary widely by molecular weight, charge density, and the extent of their association with fibers or fines. Some migration of additives within the sheet may occur, but the extent of this migration has not been studied to date.

Z-direction distribution of starch has been reported, with examples from size press treated and foam-applied starch documented. This method involves cross cutting the sheet, then spraying the cut edge with an iodine solution. The higher starch concentration areas appear as dark blue or brown and can be reported qualitatively or quantified by image analysis as depth of penetration or as a starch concentration distribution.

Dye may be added with functional additives applied via foam and for sprayed additives or even size press and coater applied additives, at least in laboratory or pilot environments. However, because dye molecules may be much smaller than strength additives of interest, and may have a different charge, this technique has some intrinsic limitations. Further challenges exist for measuring distribution of additives that are added in amounts well below 1 wt. %. In particular, synthetic strength agents are typically added in an amount of from 0.1-0.4% dry solids, compared to 1-2% starch and up to 25% of inorganic fillers.

Methods for adding products to a fibrous substrate beyond the traditional wet-end addition of chemicals have recently become of interest. Particularly, recent development of foam addition processes has highlighted the need for methods that can more effectively show the distribution of chemical additives in the z-direction of the fibrous substrates. Following are descriptions of such methods that were investigated.

### Analysis Methodologies

### Tape Splitting Followed by Nitrogen Content Analysis

Several options were considered for determining the chemical distribution in the z-direction. Tape splitting followed by nitrogen content analysis was attempted first. The tape splits were successful; however the nitrogen content of the tape's adhesive, while very low, was much higher than that of the additives, and its standard deviation was too high to reliably subtract out as a blank. Removal of the tape adhesive with solvent extraction was avoided because it potentially could have extracted some paper additives, also.

### Fluorescent Staining and Confocal Laser Scanning Microscopy

Fluorescent staining followed by confocal laser microscopy (CLSM) is a well-known method of visualizing chemical content, although not traditionally in the z-direction of the sheet. The fluorescent staining is not highly selective and can react with many materials, including other synthetic materials added in the wet end, such as a retention aid. Any amount of recycled material would also render the method less effective.

To conduct fluorescent staining, sheet samples cut to 2.5×2.5 centimeters (cm) were stained with two fluorescent dyes. Sulforhodamine 101 (Sigma Aldrich®, St. Louis, MO) was used to stain polyamide epichlorohydrin (PAE) resin as the additive of interest and acridine orange (Electron Microscopy Sciences®, Hatfield, PA) was used to stain the paper fibers. A self-bridge formation between the sulforhodamine 101 acid chloride anionic site and the PAE cationic site is expected to occur during staining. Each paper section was soaked in a 0.005 wt. % solution of sulforhodamine 101 for 3 minutes, rinsed (approximately 5 seconds) with distilled water, then soaked in a 0.005 wt. % solution of acridine orange for 3 minutes, and rinsed (approximately 5 seconds) with distilled water. While acridine orange is not required to image the PAE, it helps to highlight the fibers' location and give good contrast in the processed images. Once staining was complete, the sample was dried at room temperature for a minimum of 24 hours prior to imaging. If the z-direction cross-sectional analysis was required, the stained samples were mounted in TBS tissue freezing embedding medium (Triangle Biomedical Sciences (TBS), Durham, NC) and sections were microtomed using a Leica® 3050 cryostat (Leica®, Wetzlar, Germany). Microtomed sections were collected and mounted on a microscope slide for CLSM imaging.

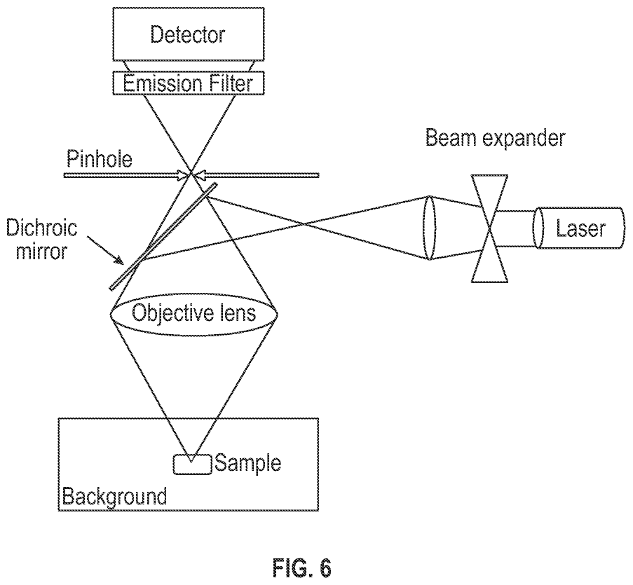

A Zeiss® 880 Confocal Laser Scanning Microscope (Zeiss®, Oberkochen, Germany) was used to study the location of the PAE resin in the sheet. CLSM is a non-destructive imaging technique that obtains an image by line-by-line scanning of the sample with a focused laser beam. A pinhole is used to block out-of-focus light, which increases the image resolution and contrast. A depiction of the laser path is shown in FIG. 6. Fluorescent staining was performed to determine the specific location of the PAE within the paper sheet. Multiple components can be imaged simultaneously by using fluorescent tags or fluorescent stains with different excitation and emission wavelengths. A 10×/0.45 (Zeiss® Plan-Apochromat) dry objective lens was used for imaging. Laser lines at 561 nm (DPSS laser) and at 458 nm (argon laser) were used because these wavelengths correspond with the emission wavelengths for sulforhodamine 101 and acridine orange. Z-stacks, which are a collection of optical sections that can be reconstructed as a 3D image, of each paper sheet were acquired. Images were processed in either the Zeiss® Zen software or Image J. (National Institute of Health, Bethesda, MD).

FIG. 6 is an example depiction of the laser path used in a confocal laser scanning microscope. Mirrors tilt the laser beam in the x and y direction to the focus plane of the objective lens, which focuses the beam onto the sample. Once the laser beam reaches the sample, the fluorescent (or reflective) light will pass back through the objective lens and the dichroic mirror reflects the light back through the pinhole to the detector.

### Layer Sectioning and Nitrogen Analysis

Microtoming equipment, as described above for use for cutting perpendicular to the surface of the sheet during investigation of the fluorescent staining method, was used to cut layers parallel to the surface of the sheet. The layers were collected and analyzed for nitrogen. The procedure details are discussed next. This method is quantitative and less subject to visual bias.

Samples were cut to approximately 0.75×0.75 cm. A sample holder was prepared by adding to it several drops of TBS tissue freezing medium and then placing it in a Leica® CryoJane® CM3050 S cryostat (Leica®, Wetzlar, Germany) held at −25° C. A fresh disposable steel blade with a polytetrafluoroethylene (PTFE) non-stick coating was used for each sample. The blade was manually adjusted to just touch the paper sample for the initial slice. The slicing depth was standardized, at 40 micrometers (um) for this particular trial although it is to be appreciated that different slicing depths are possible. After a preparatory cut, slicing proceeded quickly through the depth of the sheet until the sample medium was reached, usually after 9-13 slices. Each sectioned sample was then analyzed for nitrogen content.

Nitrogen analysis was conducted using an Antek MultiTek® (Antek, North Arlington, NJ). First, each layer weight was individually recorded to the nearest 0.001 milligram (mg). Each layer was then placed in a glass sample boat prior to loading and nitrogen analysis. The instrument destructively analyzes the sample for nitrogen via pyro-chemiluminescence, where the sample is oxidized at approximately 1000° C., forming nitric oxide from nitrogen-containing compounds. The NO is then reacted with ozone to produce NO₂ and light. The light is amplified and detected by a photomultiplier tube, which is then converted to “nitrogen counts” by the software, which directly corresponds to the base level of nitrogen in the layer plus any added nitrogen from the chemical addition process. Nitrogen count for each layer is divided by the mass to determine nitrogen concentration of the layer. For each condition, three samples were sliced and tested separately, then averaged.

### Evaluation of Results from Analysis Methodologies

The preparation of fibrous substrates or sheets and the results for each method are discussed. The fluorescent staining and CLSM method results are represented by qualitative images, while the layer sectioning and nitrogen analysis produces quantitative plots.

### Fluorescent Staining of Paper Samples and Confocal Laser Scanning Microscopy (CLSM)

The method was conducted on a set of handsheets prepared from bleached fibers of a 70% hardwood to 30% softwood ratio. No additives were used beyond foam treatment of the handsheets immediately after formation and prior to vacuum, pressing, and drying. A blank handsheet with no foam treatment was prepared for comparison. The remaining two wet sheets were foam coated with a synthetic PAE resin at a dose of approximately 0.2% of final sheet mass. Foam at roughly 300 grams per liter (g/L) density was applied to the surface of these wet handsheets in a 0.4 millimeter (mm) layer using our proprietary wet handsheet foam coating equipment and protocols as described in US Patent Pub. No. 2019/0368122. One of these foam-treated sheets was air dried as a method check. All the PAE resin should be right at the top of this sheet after drying. The other wet sheet was foam coated in a similar way, then vacuum was applied to pull the foam into the sheet and simulate the function of vacuum boxes on a paper machine. This sheet was then pressed and dried. All three sheets were then subjected to the fluorescent staining method, and the results showed PAE in red and fibers in green. The untreated handsheet showed no red, indicating that the staining/microscopy method will work well for this sample. A processed scan of the foam-coated, air-dried handsheet cross section, with the top of the sheet being on the left side of the image, showed significant red on the left side with very little red after a noticeable bondry between the red and green. This shows the PAE mostly remained at the top of the sheet. A processed scan of the foam-coated, pressed and dried handsheet cross section, with the top of the sheet being on the left side of the image, showed a gradient of red with more red on the left side and a gradual decrease in red when moving toward the right side. Once again, PAE shows as red, and fibers show as green. The image showed the PAE is present throughout the sample, although more highly concentrated in the top half of the handsheet.

The fluorescent staining and CLSM method worked well for these handsheets. Although the images are qualitative only, they do show the first indication of the chemistry's location. However, there are some concerns with this method. Since the sheet is rewetted during the application of the dye, it is not known if any added PAE has the potential for partial migration; that is, after the sheet is dried a second time after staining, the final chemical distribution may be altered from its initial state.

When applied to more realistic sheets that also contained commonly-used wet end additives such as retention aids and cationic starch, we found these other additives also attracted the stain intended for the strength aid. This masked the distribution of the foam-applied chemistry, so the method failed. Some modifications, such as fluorescent tagging or other modifications to the PAE prior to foam application, were considered, but were ultimately not investigated due to high cost and concern over potential impacts on the chemical distribution pattern. A method that tested finished sheets without rewetting and that used unmodified, commercially available materials was desired.

### Layer Sectioning and Nitrogen Analysis

The sheets prepared for this analysis were multi-ply (the plies had different furnish compositions). The foam density was approximately 150 g/L and contained a foaming agent and a synthetic dry strength agent (DSA). The foam was applied between the first and second layers with the goal of providing local strength at and around the ply zone and increasing the strength of the second ply. An increase in z-direction strength performance indicated success and contributed to our understanding of how to control chemical penetration.

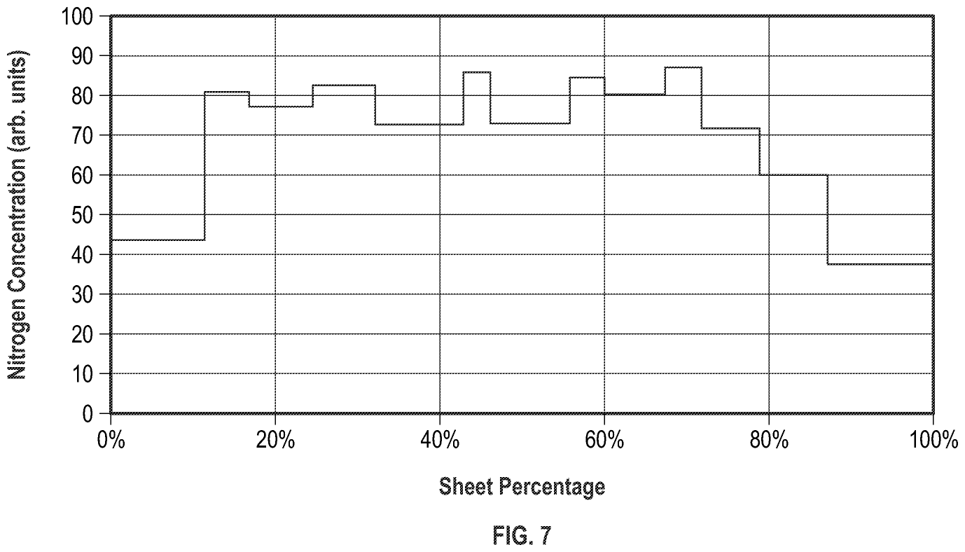

Multi-ply sheets with no foam treatment and additional sheets with foam-applied 0.4 and 0.8 weight % DSA were prepared. The mass of each microtome layer was divided by the sum of all the layers and the nitrogen data was presented as sheet mass percentage (0% is the sheet bottom and 100% is the top). The raw nitrogen counts of each layer are divided by the layer mass and a normalization factor of 2,000,000 to lower the scale. For example, a single untreated sample's plot is shown in FIG. 7.

FIG. 7 shows a single sample's data from an untreated multi-ply sheet after the layering and nitrogen analysis was completed and the data was normalized. The nitrogen concentration is expressed in terms of nitrogen counts/mg and normalized by a factor of 2,000,000 to lower the scale. The sheet percentage indicates the depth into the sheet, with 0% being the bottom of the sheet and 100% being the top. The relative DSA concentration for each of 13 layers is distinctly visible.

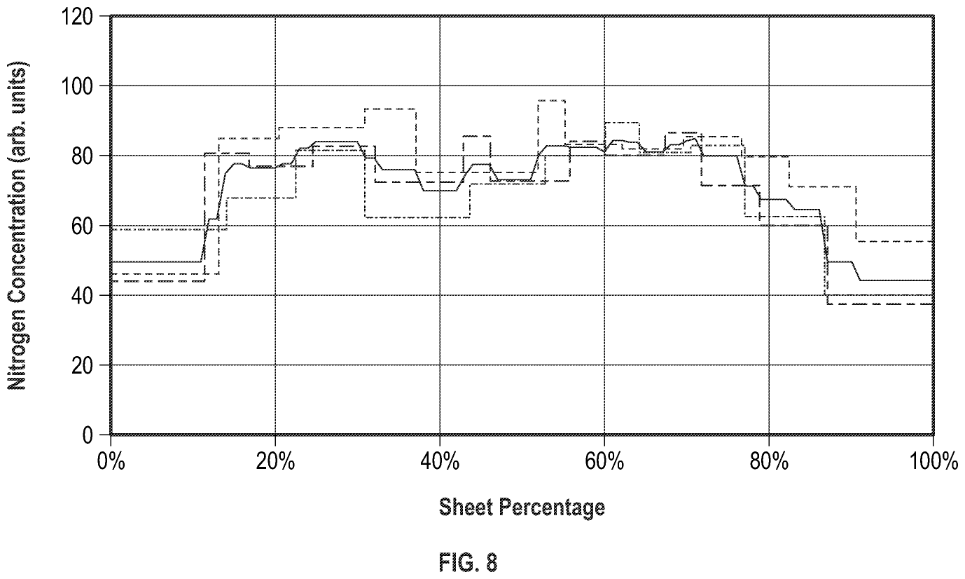

Recognizing the potential sampling bias that a single preparation might incur, two additional samples for each condition were prepared in an identical manner, as previously described. Using the sheet percentages, the samples were averaged together at each point, to provide a reasonable average composite for a single condition. This is shown in FIG. 8.

FIG. 8 shows the composite of three samples for a single untreated multi-ply sheet, where the smoother line with the more gradual changes in the Y direction is the composite line. Each individual sample's data from layer sectioning and nitrogen analysis is shown in the lines with sharp, abrupt changes in the Y direction. Variability was relatively low for this sheet, as is apparent from the small differences between the lines. The average value of the three individual samples is averaged together at each percentage point and plotted as the composite line.

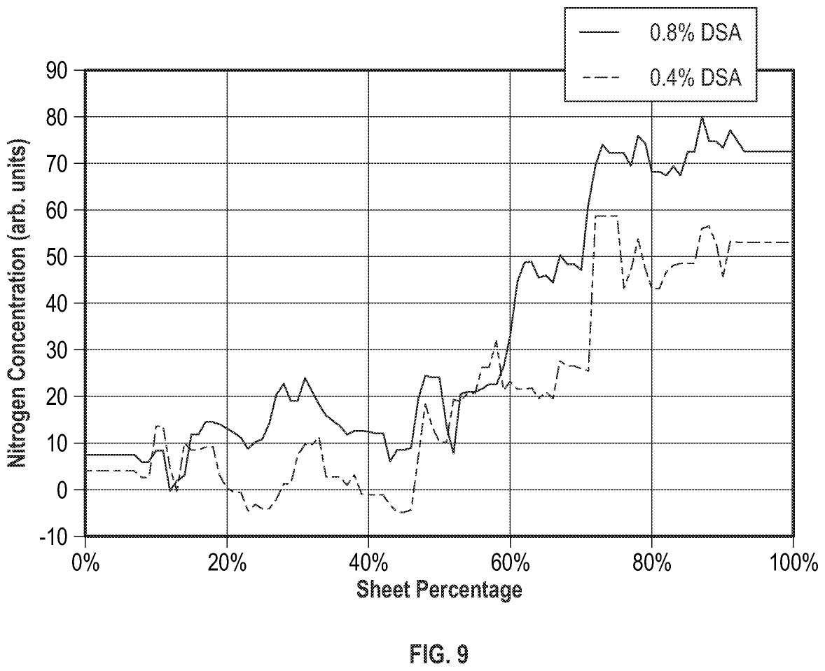

Next, the foam-treated multi-ply sheets were analyzed. The sample data for both foam-treated conditions, with 0.4 and 0.8 weight % added DSA, were treated identically. The results in FIG. 9 are shown with the average background nitrogen counts subtracted from the foam-treated results. This data treatment removes the background nitrogen counts and provides a plot that represents the foam-applied DSA's distribution within the sheet. Some “negative” concentrations are observed, which indicate that the nitrogen concentration in the treated sheet was just below that of the blank at that location. This is to be expected with natural variations—realistically, this can be interpreted to mean a negligible amount of DSA was present at that location in the sheet.

FIG. 9 shows the distribution of added DSA within two foam-treated sheets: a 0.4%-dosed sheet and a 0.8%-dosed sheet. Concentration of the chemistry is shown to be lower for the 0.4% condition, as expected, but the two conditions follow similar chemical distribution patterns. Since the top ply accounts for approximately 23% of the sheet, the foam was added at 77% of the way through the sheet. DSA concentration is highest at and around this point for both conditions, as expected.

### EXAMPLE DISCUSSION

The fluorescent staining and CLSM analysis method was the first successful attempt at imaging the distribution of foam-applied chemistry in the z-direction of the sheet. However, this conditions are most ideal for this method in laboratory-produced handsheets with no other added chemistry. Attempts to analyze sheets with wet-end additives, as is ubiquitous in the industry, are hindered because of a strong stain response with other components in the sheet. Thus, it is difficult to separate the foam-applied additives from the background. This method is also primarily visual and not quantitative.

The cryostat layer sectioning and nitrogen analysis method is much more robust than fluorescent staining/CLSM and quantifies the chemical distribution, particularly for foam or spray-applied additives. By subtracting the nitrogen content of the background sheet (i.e., the reference sheet mentioned above), it is possible to view a treated sheet's DSA distribution. In embodiments, analysis of the chemical distribution can be paired with strength tests, with the results used to modify parameters of the application process if desired. For example, the location at which a sheet splits during z-direction strength testing (e.g., ZDT or Scott bond) can be compared with the chemical distribution results. This may help determine the needed DSA dose and foam application parameters required to attain a target.

In FIG. 9, the chemical distribution of two sheets with different doses of a foam-applied DSA are compared. The chemical distribution is similar between the two, showing that most of the applied chemistry penetrates only to approximately halfway through the finished sheet, with the highest concentration in both cases around the application point between the two plies (77% through the sheet). Interestingly, the chemistry not only moved down into the sheet (the direction of the vacuum), but also migrated to the top 23% of the sheet—the top ply contains a high concentration of chemistry.

A ratio of the total applied chemistry between the two conditions can be approximated by integrating under the curve and comparing the total area for each condition. If the retention were identical, then exactly half the area would be expected for the 0.4% DSA condition versus the 0.8% DSA condition. However, separate analysis indicated retention of 68% for the 0.8% DSA condition and 79% for the 0.4% DSA condition. Higher retention is expected for lower dosage rates, as commonly seen in the industry. Integration results and retention values are shown in Table 1.

Table 1. Area under the curve for both conditions in FIG. 9 and the conditions' separately analyzed retention results. An “expected area” is shown for the 0.4% DSA condition by dividing the 0.8% DSA area in half, then multiplying by a ratio of the two retention values. The expected area differs from the actual value by only 5.5%, providing some outside validation of the results.

TABLE 1

| | 0.8% DSA | 0.4% DSA |
|---|---|---|
| Value | condition | condition |
| Area under | 3462 | 2144 |
| the curve, | | |
| FIG. 12 | | |
| Retention | 68% | 79% |
| Expected area | — | 2025 |

Initial conclusions made by direct observation of FIG. 9 were that the pattern of chemical distribution was similar between the two and that the 0.4% DSA condition likely yielded higher retention, since its value does not appear to be half that of the higher dosage condition along the length of the curve. As shown in the table, the integration of the plots matches reasonably well with separate retention analyses. This confirms the second conclusion and provides some support for the first—if the patterns were wildly different than pictured, the retention analysis would most likely not corroborate the integration.

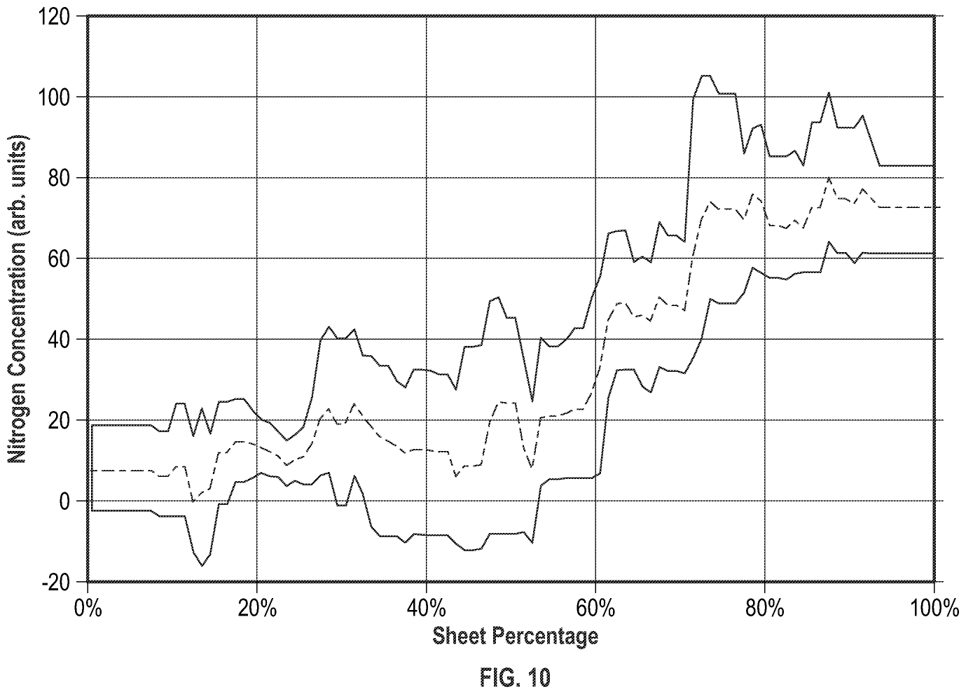

Nine samples were tested from a single sheet with 0.8% DSA applied via foam, and were tested for nitrogen counts, rather than the typical three samples, to investigate method reproducibility. The highest three nitrogen concentrations from any of the samples at every point throughout the sheet (0-100%) were averaged. Eight of the nine samples contributed to a maximum at some point along the curve. This process was repeated for the lowest three concentrations at every point to give the minimum possible result. This approach, when plotted in FIG. 10, shows the range of possible outcomes for this method. Out of 84 possible combinations from the nine samples tested, any combination of three samples would lie within the area between the solid lines in FIG. 10. The average outcome, obtained by averaging all nine samples, is shown as a dashed line between the solid lines. The range-of-outcomes plot sufficiently demonstrates that this method can consistently produce a similar pattern regardless of sampling error.

FIG. 10 shows a range of possible outcomes for the 0.8% DSA condition when using this method. Nine samples were used to generate this plot, three times the typical number. An average of any three samples would produce a line within the area between the solid lines, with the dashed line representing the average of all nine points. As seen, the method consistently produces a very similar pattern regardless of the sample combination.

The layer sectioning and nitrogen analysis method is preferably conducted under conditions where thickness of the sheet under evaluation is substantially larger than the practical layer thickness so as to provide at least 3 layers for analysis. In embodiments, the layer depth is 40 microns although it is to be appreciated that alternative layer depths are possible. Analysis of a sheet with a thickness of at least 3 times the practical layer thickness, e.g., at least 120 microns or more, is desirable to yield useful data. Impact of freezing minimum on the measurements may be minimized by subtraction of background correction of the final chemical distribution results because the effect will be present in both the untreated and treated sheets. Sufficient internal bond strength of the sheets under evaluation is desired to avoid crumbling during slicing of the sample so as to produce cleanly sliced sheets. If the sheet crumbles, some material may be lost prior to collection and nitrogen analysis, and the sections will not be as clearly defined.

In embodiments, directionality of the slicing method—whether top-down or bottom-up—may influence the results. Sheet slices may be more uniform and intact when the first section comes from the bottom of these samples, which may lead to more reliable results. When using the method on a novel sheet, it may be desirable to obtain sections from both orientations initially to determine which is more uniform and intact.

In embodiments, the method includes calibrating the nitrogen concentration to the actual chemical concentration. The results described herein are nitrogen concentrations (nitrogen counts/mg), but this can be combined easily with retention analysis at a known dose to determine an actual chemical concentration at any point in the sheet.

### EXAMPLE CONCLUSIONS

The fluorescent staining and CLSM method offers a quick and reliable way to check the chemical distribution visually when an appropriate sheet is used. This method is more suitable for laboratory-produced handsheets, but the technique can be used to help refine understanding of the foam application process and how its variables can affect the chemical distribution.

The layer sectioning and nitrogen analysis method can generate a plot of added DSA concentration through the z-direction of the sheet. This method should be useful in a range of applications, including commercially generated paper products. Chemical distribution results can give additional meaning to strength tests and aid with improving and understanding additive addition processes, particularly for foam-assisted or spray additive addition processes. This method visualizes the chemical distribution after subtracting any interference from the base sheet, and it can provide meaningful differentiation between similar treatment conditions. This method should be broadly applicable to a variety of potential additives and substrates, provided the additives contain detectable amounts of nitrogen.

While at least one exemplary embodiment has been presented in the foregoing detailed description, it should be appreciated that a vast number of variations exist. It should also be appreciated that the exemplary embodiment or exemplary embodiments are only examples, and are not intended to limit the scope, applicability, or configuration of the disclosure in any way. Rather, the foregoing detailed description will provide those skilled in the art with a convenient road map for implementing the exemplary embodiment or exemplary embodiments. It should be understood that various changes can be made in the function and arrangement of elements without departing from the scope of the disclosure as set forth in the appended claims and the legal equivalents thereof

What is claimed is:

1. A method of preparing paper, the method comprising the steps of:
applying a paper additive to a first sheet of paper using an initial production technique;
sectioning the first sheet of paper into a first section and a second section, wherein the first sheet of paper has a top surface defined in an X-Y plane, wherein a Z axis is perpendicular to the X-Y plane, and wherein the first section and the second section are defined at different positions along the Z axis;
measuring a paper additive concentration in the first section and in the second section; and
adjusting the initial production technique to a subsequent production technique to influence a paper additive concentration profile along the Z axis.

2. The method of claim 1, further comprising:
applying the paper additive to a second sheet of paper using the initial production technique, where the same initial production technique is used for the first sheet of paper and the second sheet of paper; and
measuring a selected paper property of the second sheet of paper, wherein the paper additive influences the selected paper property.

3. The method of claim 2, wherein the selected paper property is strength, and wherein the paper additive is a paper strength additive.

4. The method of claim 1, further comprising:
attaching the first sheet of paper to a microtome, wherein the microtome is configured to slice the first sheet of paper perpendicular to the Z axis.

5. The method of claim 4, wherein the microtome is a cryostat microtome, and wherein attaching the first sheet of paper to the cryostat microtome comprises freezing an attachment fluid positioned between the cryostat microtome and the first sheet of paper.

6. The method of claim 5, wherein the attachment fluid comprises a tissue freezing medium.

7. The method of claim 1, wherein the paper additive comprises nitrogen, wherein measuring the paper additive concentration comprises measuring a nitrogen concentration in the first section, and determining the paper additive concentration in the first section based on the nitrogen concentration of the first section and a percentage of a paper additive molecular weight from nitrogen.

8. The method of claim 7, wherein the nitrogen concentration is measured using pyro-chemiluminescence.

9. The method of claim 7, wherein measuring the paper additive concentration further comprises:
producing a reference sheet of paper using a reference production technique, wherein the reference production technique is the same as the initial production technique with the exception that the reference production technique is free of a paper additive addition step, such that the reference sheet of paper is free of the paper additive;
measuring the nitrogen concentration in the reference sheet of paper;
determining a paper additive induced nitrogen concentration in the first section by reducing the nitrogen concentration measured in the first section to account for the nitrogen concentration measured in the reference sheet of paper; and
determining the paper additive concentration based on the paper additive induced nitrogen concentration in the first section.

10. The method of claim 1, further comprising:
repeating the method of claim 1, with the exception of applying the paper additive to a subsequent sheet of paper using the subsequent production technique, wherein the paper additive concentration is measured in the subsequent sheet of paper; and
comparing the first sheet of paper to the subsequent sheet of paper to inform adjustments to an improved paper production technique.

11. The method of claim 1, wherein:
sectioning the first sheet of paper comprises sectioning the first sheet of paper into more than two sections.

12. The method of claim 1, wherein:
applying the paper additive to the first sheet of paper comprises applying the paper additive to a surface of the first sheet of paper.

13. A method of analyzing a sheet of paper, the method comprising the steps of:
attaching a first sheet of paper to a microtome, wherein the first sheet of paper comprises a paper additive, wherein the first sheet of paper has a top surface defined in an X-Y plane, and wherein the microtome is configured to slice the first sheet of paper along a Z axis, wherein the Z axis is perpendicular to the X-Y plane;
slicing the first sheet of paper into a first section and a second section with the microtome, wherein the first section and the second section are defined at different positions along the Z axis; and
measuring a paper additive concentration in the first section and in the second section.

14. The method of claim 13, wherein the microtome is a cryostat microtome.

15. The method of claim 14, wherein attaching the first sheet of paper to the cryostat microtome comprises freezing an attachment fluid positioned between the cryostat microtome and the first sheet of paper.

16. The method of claim 15, wherein the attachment fluid comprises a tissue freezing medium.

17. The method of claim 13, wherein measuring the paper additive concentration comprises:
measuring a nitrogen concentration in the first section, wherein the paper additive comprises nitrogen; the method further comprising
determining the paper additive concentration in the first section based on the nitrogen concentration of the first section and a percentage of a paper additive molecular weight from nitrogen.

18. The method of claim 17, wherein the nitrogen concentration is measured using pyro-chemiluminescence.

19. The method of claim 17, wherein measuring the paper additive concentration further comprises:
producing a reference sheet of paper using a reference production technique that is the same as an initial production technique used for the first sheet of paper with the exception that the reference production technique is free of a paper additive addition step, such that the reference sheet of paper is free of the paper additive;
measuring the nitrogen concentration in the reference sheet of paper;
determining a paper additive induced nitrogen concentration in the first section by reducing the nitrogen concentration measured in the first section to account for the nitrogen concentration measured in the reference sheet of paper; and
determining the paper additive concentration based on the paper additive induced nitrogen concentration in the first section.

20. A method of preparing paper, the method comprising the steps of:
applying a paper additive to a first sheet of paper using an initial production technique;
applying the paper additive to a second sheet of paper using the initial production technique, where the same initial production technique is used for the first sheet of paper and the second sheet of paper;
attaching the first sheet of paper to a microtome, wherein the first sheet of paper has a top surface defined in an X-Y plane, and wherein the microtome is configured to slice the first sheet of paper along a Z axis, wherein the Z axis is perpendicular to the X-Y plane;
slicing the first sheet of paper into a first section and a second section with the microtome, wherein the first section and the second section are defined at different positions along the Z axis;
producing a reference sheet of paper using a reference production technique, wherein the reference production technique is the same as the initial production technique with the exception that the reference production technique is free of a paper additive addition step, such that the reference sheet of paper is free of the paper additive;
measuring a nitrogen concentration in the reference sheet of paper;
measuring the nitrogen concentration in the first section;
determining a paper additive induced nitrogen concentration by reducing the nitrogen concentration measured in the first section to account for the nitrogen concentration measured in the reference sheet of paper;
determining a paper additive concentration in the first section based on the paper additive induced nitrogen concentration;
measuring a selected paper property of the second sheet of paper, wherein the paper additive influences the selected paper property;
repeating the above steps, with the exception of applying the paper additive to a subsequent sheet of paper using a subsequent production technique different than the initial production technique, wherein the paper additive concentration is measured in the subsequent sheet of paper; and
adjusting an improved paper production technique based on comparisons of the first sheet of paper to the subsequent sheet of paper.
